SEQUENCE LISTING (12 sequences)

SEQ ID NO: 1 - RNA, 68 nt, Hepatitis D virus
aacaugcuucggcauggcgaacgugggacgucccagccuccucgcuggcgccggcuggggccggcaug

SEQ ID NO: 2 - RNA, 37 nt, Homo sapiens
cugaugaguccgugaggacgaaacgaguaagcucguc

SEQ ID NO: 3 - DNA, 55 bp, Homo sapiens
ctgcttgccgcctccctgccggcaggttacttctgatgagtccgtgaaggacgaa

SEQ ID NO: 4 - DNA, 54 bp, Homo sapeans
  misc_feature (26)..(26): n is a, c, g, or t
ttcggcatggcgaatgggaccctgcntaggcaggccagacctcatcccagcaag

SEQ ID NO: 5 - DNA, 32 bp, Homo sapeans
cacatccctggagtctgaaaccactgagggtt

SEQ ID NO: 6 - DNA, 27 bp, Homo sapeans
gtgtagggacctcagactttcctgact

SEQ ID NO: 7 - DNA, 32 bp, Homo sapeans
cacctcacagacccatcgatggccaacggtgc

SEQ ID NO: 8 - DNA, 26 bp, Homo sapeans
gtggagtgtctgggtagctaccggtt

SEQ ID NO: 9 - DNA, 38 bp, Homo sapeans
cacatccctggagtctgaaaggacaacggtgctgggaa

SEQ ID NO: 10 - DNA, 90 bp, Homo sapeans
gggtaccgagctcgaatttccagacacatccctggagtctgaaaggacaacggtgctgggaacattctaggagggtgatgggacagaggg

SEQ ID NO: 11 - DNA, 29 bp, Homo sapeans
cacatccctggagtctgaaaggactgagg

SEQ ID NO: 12 - DNA, 32 bp, Homo sapeans
acagacccatcgatggccaacggtgctgggaa

(19) United States
(12) Patent Application Publication
(10) Pub. No.: US 2023/0212604 A1
(43) Pub. Date: Jul. 6, 2023

(54) METHODS AND COMPOSITIONS FOR EFFICIENT AND PRECISE GENE EDITING IN MAMMALIAN BRAIN TO PREVENT OR TREAT NERVOUS SYSTEM DISORDERS

(71) Applicant: Board of Supervisors of Louisiana State University and Agricultural and Mechanical College, Baton Rouge, LA (US)

(72) Inventors: Xiao-Hong LU, Shreveport, LA (US); Xinli TIAN, Shreveport, LA (US)

(73) Assignee: Board of Supervisors of Louisiana State University and Agricultural and Mechanical College, Baton Rouge, LA (US)

(21) Appl. No.: 16/711,409

(22) Filed: Dec. 11, 2019

Related U.S. Application Data
(60) Provisional application No. 62/778,100, filed on Dec. 11, 2018.

(51) Int. Cl. C12N 15/86; C12N 9/22; C12N 15/11; A61K 38/46; A61K 31/7105

(52) U.S. Cl. CPC C12N 15/86; C12N 9/22; C12N 15/11; A61K 38/465; A61K 31/7105; C12N 2310/20; C12N 2800/80; A61K 48/00

(57) ABSTRACT

A method for gene editing in a vertebrate brain comprising: intravascular administration of a brain penetrable viral vector including a target sequence in a genomic locus of interest and a CRISPR enzyme; genomic integration via Non-Homologues End Joining (NHEJ) in post-mitotic neurons; and editing a monopartite cell-type specific gene via NHEJ knock-in a sgRNA flanked by self-cleaving ribozymes into 3′UTR to use an endogenous promoter for sgRNA expression.

### CROSS REFERENCE TO RELATED APPLICATIONS/PRIORITY

The present invention claims priority to U.S. Provisional Patent Application No. 62/778,100 filed Dec. 11, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

The discovery of the genetic basis of hereditary disorders led to the early concept of gene therapy in which “exogenous good” DNA be used to replace the defective DNA. Currently, researchers have realized that the simple idea of gene replacement is actually much more challenging and technically complex to implement both safely and effectively, especially for gene therapy in central nervous system (CNS). Three major hurdles were present and unresolved by current technology: delivery, low efficiency in post-mitotic neurons, and lack of cell type specificity.

Delivery: properties of genome editing nucleases, including large size, negative charge, limited membrane penetrating, weak tolerance for serum, and low endosomal escape, have limited their application in therapeutic genome editing. The situation is even worse in the case of therapeutic gene editing for CNS disorders. Currently, Adeno-associated viruses vectors (AAVs) has been commonly used for in vivo gene delivery due to potential low immunogenicity and relatively low site-specific integration. However, largely because of its low capability to cross the blood-brain barrier, there has been only limited success in delivering AAVs and their genetic cargo to the CNS.

Low efficiency in post-mitotic neurons: CRISPR-Cas9 induces DNA double-strand breaks (DSBs) at single-guide RNA (sgRNA)-specific loci in the genome, which are repaired through either NHEJ or HDR pathways. While NHEJ introduces an unpredictable pattern of insertion or deletion (indel) mutations, HDR directs a precise recombination event between a homologous DNA donor template and the damaged DNA site. Thus, HDR can be used to precisely introduce sequence insertions, deletions or mutations by encoding the desired changes in the donor template DNA. While HDR-based genome editing has been demonstrated to be useful for precise genome editing, application of HDR-based genome editing has been limited to mitotically dividing cells. This is because HDR had previously been found to occur primarily in the S and G2 phases of the cell cycle in mitotically dividing cells. In fact, HDR was found to occur rarely in postmitotic cells such as neurons. Thus, in the brain, HDR-based genome editing has been performed previously only in dividing cells such as neuronal progenitors in the embryo. Therefore, precise genome editing has been a challenge in postmitotic cells.

Cell type specificity: The third major hurdle for CNS gene editing is neuronal cell-type specificity. CNS is comprised of a heterogeneous population of cells, including different neuronal subtypes and glial cells. Due to this complexity, genetic manipulations that affect distinct populations of cells often yield different results. In order to introduce transgenes into unique cellular populations, modern genetics has enabled even more precise cellular specificity by incorporating cell-type-specific promoters or site-specific recombinase to introduce transgenes into unique cellular populations. However, except a few exceptions (e.g. bacterial artificial chromosome (BAC) transgenesis), most of the promoters used to drive the transgene expression in AAV or traditional transgenesis are not specific. Cre-LoxP recombination is one of the site-specific recombinase technologies that allows DNA modification to be targeted to a specific cell type or be triggered by a specific external event. While easier to control than homologous recombination, the Cre-lox system was less efficient as the genetic distance increased between loxP sites. Furthermore, such two-patriate genetic system has been presented as almost impossible to implement in human therapeutic gene editing.

Despite the life and death benefits of resolving these challenges, they stubbornly remained.

### SUMMARY

Wherefore, it is an object of the present invention to overcome the above-mentioned shortcomings and drawbacks associated with the current technology.

According to one embodiment, in order to overcome the three major hurdles of therapeutic gene editing in CNS: Delivery, low efficiency in post-mitotic neurons, and lack of cell type specificity, this invention involves novel methods and compositions of gene editing in mammalian brain to prevent or treat nervous system disorders. One embodiment of the presently disclosed invention relates to a method for gene editing in adult mammalian brain via intravascular administration of a brain penetrable Adeno-Associated Virus (AAV). A further embodiment of the presently disclosed invention relates to methods and toolsets for efficient and cell-type-specific genome editing as a therapy to treat brain disorders, such as neurodevelopmental, neurodegenerative, cerebrovascular, and psychiatric disorders. A further embodiment of the presently disclosed invention relates to efficient and precise genomic integration (replacement) via non-homologues end joining (NHEJ) in post-mitotic neurons. A still further object of the invention is to use a non-compatible split the protospacer adjacent motif and gRNA recognition sequence to facilitate directional transgene integration into human genome. The original gRNA targeting and protospacer adjacent motif sequences are destroyed for reducing the off-target effects. A further embodiment of the presently disclosed invention relates to monopartite cell-type specific gene editing via non-homologues end joining knock-in a sgRNA flanked by self-cleaving ribozymes at 3′UTR for monopartite cell type specific gene editing. A further embodiment of the presently disclosed invention relates to a genetic strategy and fluorescence mouse line as a gene-editing reporter for preclinical studies of the efficiency of CRIPSR/Cas9 mediated gene editing. A further embodiment of the presently disclosed invention relates to a genetic strategy and fluorescence mouse line as a Cre-independent single-neuron genetic labelling and manipulation to visualize neuronal/glia cell morphology, neurodegeneration, neurodevelopment for preclinical CNS drug discovery. A further embodiment of the presently disclosed invention relates to a Cre independent CRIPSR/Cas9 gene editing reporter to facilitate the preclinical studies of the efficiency of CRIPSR/Cas9 mediated gene editing.

The invention relates to methods for gene editing in a vertebrate brain comprising intravascular administration of a brain penetrable viral vector including a target sequence in a genomic locus of interest and a CRISPR enzyme, genomic integration via Non-Homologues End Joining (NHEJ) in post-mitotic neurons, and editing a monopartite cell-type specific gene via NHEJ knock-in a sgRNA flanked by self-cleaving ribozymes into 3′UTR to use an endogenous promoter for sgRNA expression. According to a further embodiment, the brain is an adult mammalian brain. According to a further embodiment, the viral vector is an Adeno-Associated Virus (AAV). According to a further embodiment, the AAV is one of AAV-PHP.eB, AAV1, AAV2, AAV3, AAV4, AAV5, AAV6, AAV7, AAV8, AAV9, AAV10, AAV11, AAV12, AAV2/9, AAV2/8, and AAV-DJ. According to a further embodiment, the CRISPR enzyme is a Cas9. According to a further embodiment, the Cas9 is one of SpCas9 and SaCas9. According to a further embodiment, the invention further comprising the steps of treating a brain defect or disorder in the vertebrate. According to a further embodiment, the brain has a genetic defect. According to a further embodiment, the genetic defect is one of mutation, copy number variation, nucleotide repeat, duplication, triplication, and delete. According to a further embodiment, the vertebrate has a nervous system disorder.

The invention further relates to methods for gene editing in a human brain comprising intravascular administration of a brain penetrable viral vector including a target sequence in a genomic locus of interest and a CRISPR enzyme, genomic integration via Non-Homologues End Joining (NHEJ) in post-mitotic neurons, editing a monopartite cell-type specific gene via NHEJ knock-in a sgRNA flanked by self-cleaving ribozymes into 3′UTR to use an endogenous promoter for sgRNA expression, and treating a brain defect or disorder in the vertebrate, wherein the viral vector is an Adeno-Associated Virus (AAV) AAV-PHP.eB, the CRISPR enzyme is one of SpCas9 and SaCas9, and the brain is one of mutation, copy number variation, nucleotide repeat, duplication, triplication, and delete genetic defect.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### Incorporation of Sequence Listing (Text File)

This application contains a text file named LSUHS_P101AUS_ST25.txt, which is 2,410 bytes (measured in MS-DOS), which was created on Dec. 14, 2021, and is hereby incorporated by reference into the specification of this application in its entirety. The text file sequence listing contains RNA and DNA sequences contained in FIGS. 6C, 7B, 8A, and 8B.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A to 1D are schematic illustrations of a method of the gene editing reporter mice, according to embodiments of the disclosed invention, and a micrograph demonstrating the efficacy of the method;

FIGS. 2A to 2Z are twenty-six micrographs demonstrating the high efficiency of CNS gene editing via the intravascular administration of brain penetrable AAV, according to one embodiment of the disclosed invention;

FIGS. 3A to 3I are nine micrographs demonstrating the usage of the gene editing reporter mice for visualization of single-neuron morphology and neurodegeneration in a stroke mouse model, according to one embodiment of the disclosed invention;

FIGS. 4A and 4B are schematic illustrations of a method of AAV mediated systemic delivery to correct a neurodevelopmental disorder related gene duplication, according to one embodiment of the disclosed invention;

FIGS. 5A to 5C are schematic illustrations of a method to use non-homologues end joining (NHEJ) for efficient and precise gene replacement (correction) in post-mitotic neurons, according to one embodiment of the disclosed invention, and two micrographs of demonstrating the usage;+

FIGS. 6A-6D are schematic illustrations of a method to knock-in a sgRNA flanked by self-cleaving ribozymes at 3′UTR for monopartite cell type specific gene editing, according to one embodiment of the disclosed invention;

FIGS. 7A-7E are PCR, sequences, and micrographs that show validation of the precise gene editing via junction PCRs and genomic sequencing to use gene editing reporter mice to visualize Drd2 expressing cells; and

FIGS. 8A to 8D are schematics illustrating the in vivo correction of a human genomic duplication associated with the neurodevelopmental disorders via intravenous delivery of gRNA and Cas9, and PCRs and genomic sequencing.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

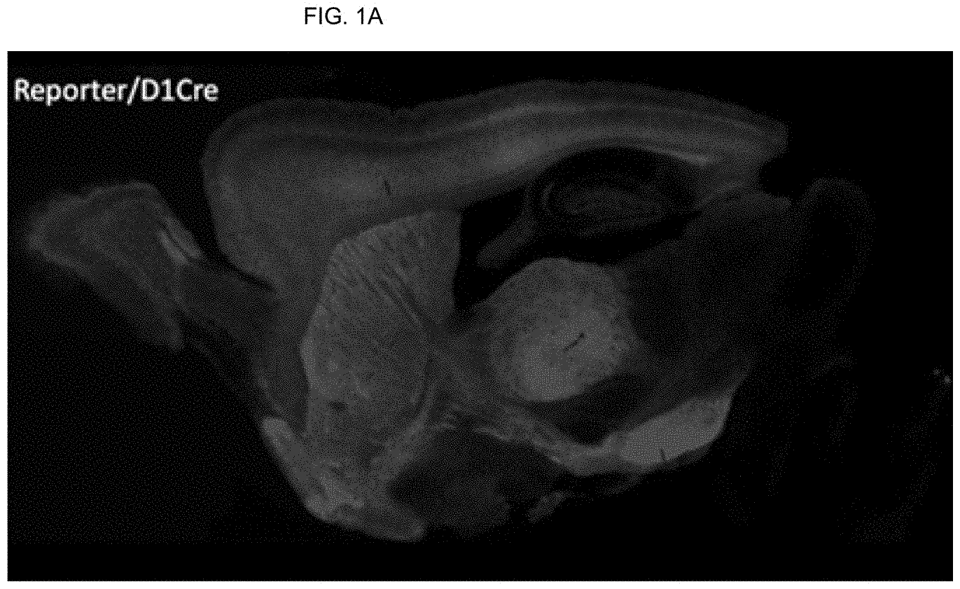

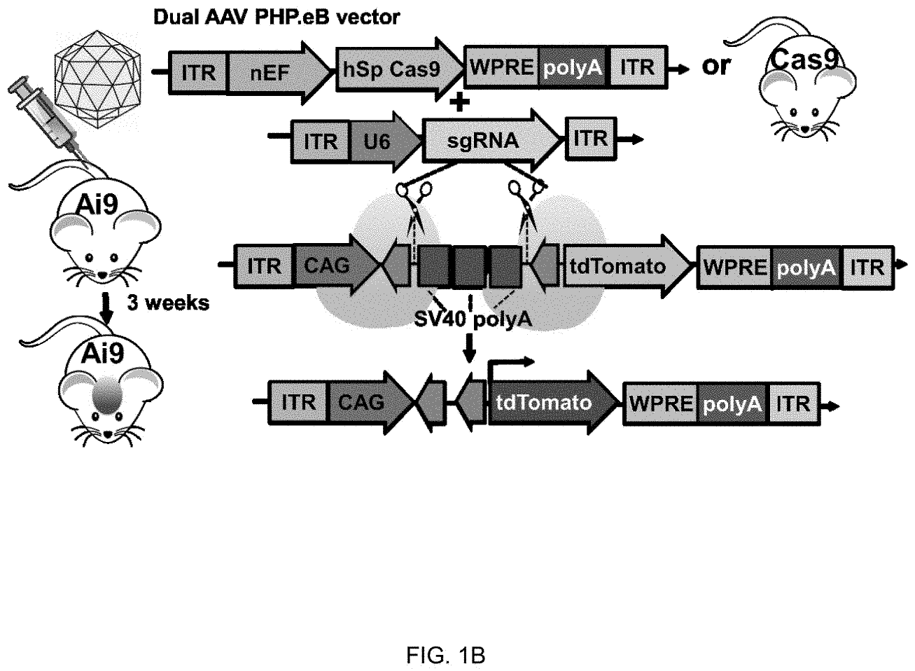

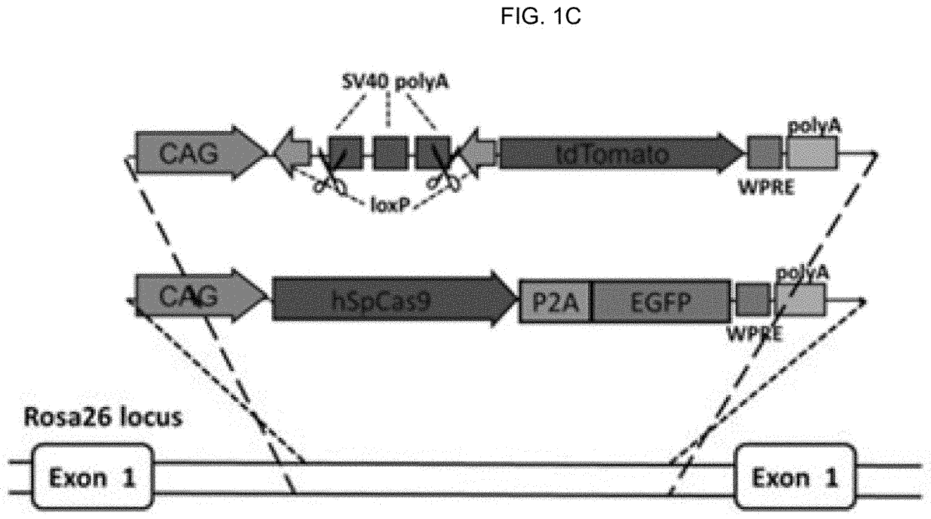

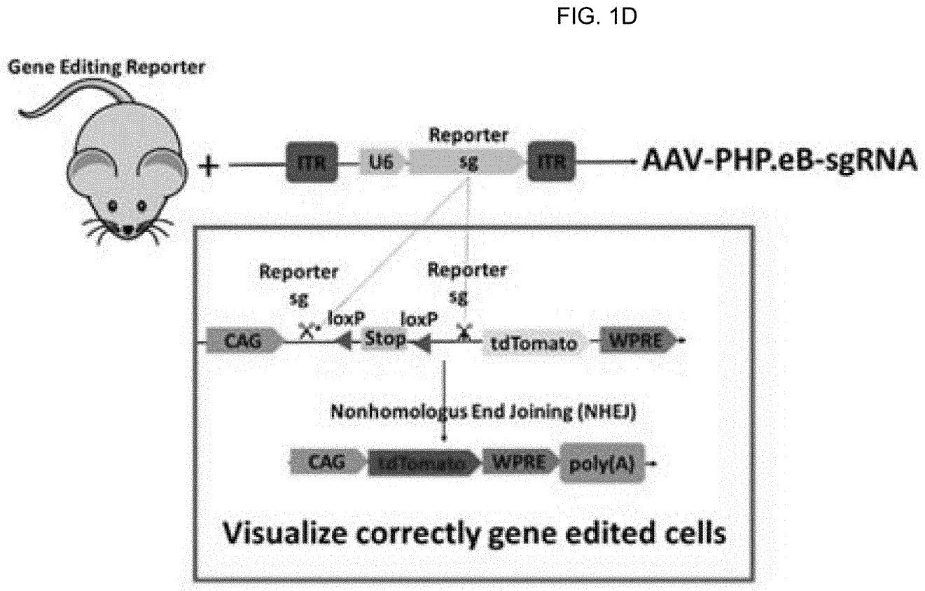

Turning now to FIGS. 1A to 8D, a brief description concerning the various components of the present invention will now be briefly discussed. To detect Cas9 activity in vivo, current methods mainly rely on genotyping and Sanger sequencing, which are inefficient and not accurate enough to detect small events of gene editing. In order to develop an animal model that can visualize the cells with Cas9 activity and correct gene editing in the CNS in vivo, the inventors have designed a genetic strategy and mouse model to report the Cas9 mediated gene editing activity. More specifically, the inventors crossed a Cre-dependent red fluorescence reporter mice (FIG. 1A, shown with crossing with Drdla—Cre mice) with the ROSA-26-hspCa9-2A-GFP mice (FIGS. 1A-1D). These mice have a ubiquitous expression of human spCas9 and a foxed STOP cassette in front of a tdTomato open reading frame. The guide RNA was designed to recognize protospacer adjacent motif (PAM) sites on either side of foxed in the genomic regions of the reporter mice. sgRNA targeting the stop cassette and multiple tandem sgRNA can be packaged into the same vector (e.g., AAV). Following Cas9-mediated double-stranded breaks (DSB) and non-homologues end joining, the floxed STOP cassette is removed, thus the expression of red fluorescence protein (FIG. 1C). Therefore, the inventors have described here a novel fluorescence mouse line as a Cre independent CRIPSR/Cas9 gene-editing reporter, which can be used for preclinical studies to report the efficiency of CRIPSR/Cas9 mediated gene editing.

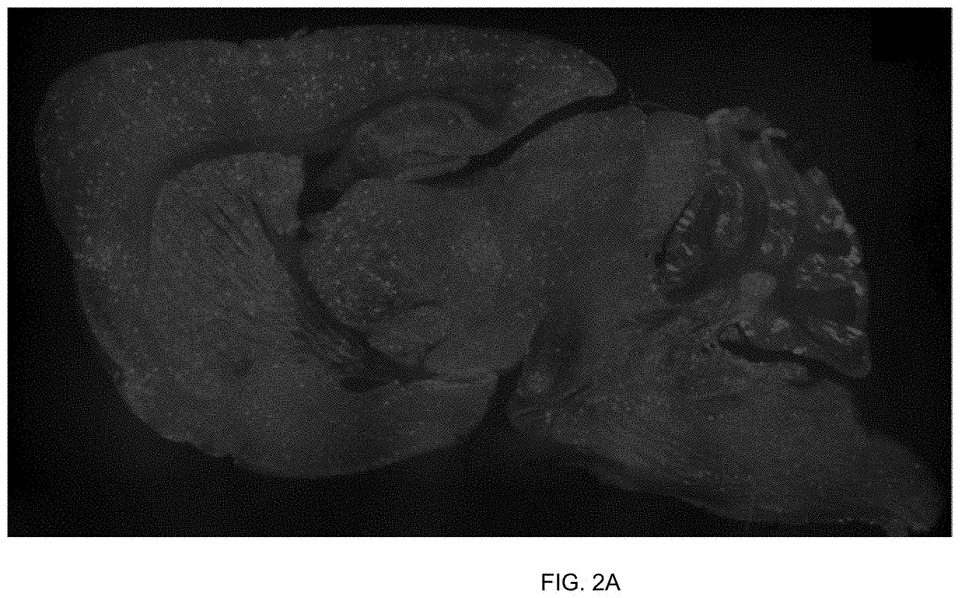

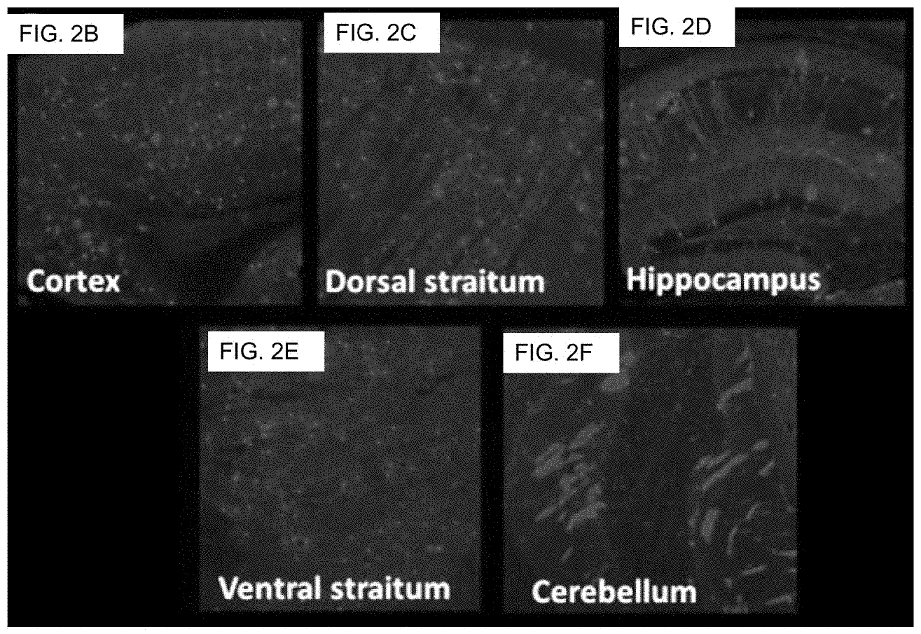

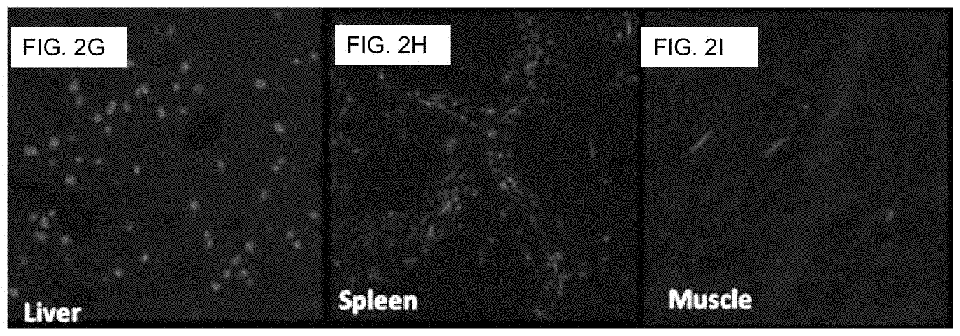

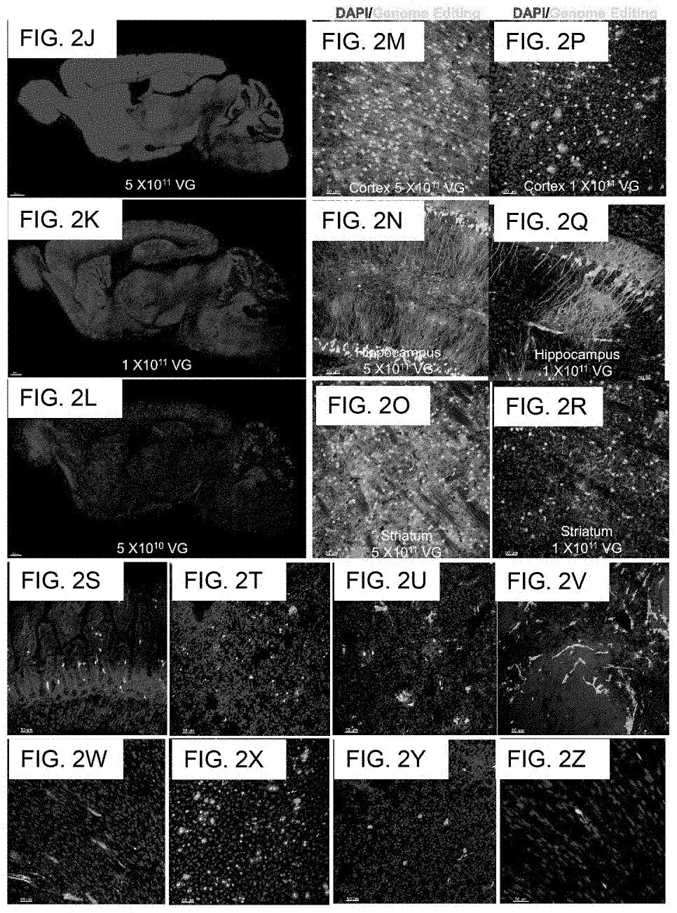

Turning next to FIGS. 2A to 2I, which is the very first realization of highly efficient gene editing in adult mammalian brain via intravascular systemic administration of a brain-penetrable Adeno-Associated Virus (AAV). ssAAV-PHP.eb: sgRNA at 1×1011 vg/mouse was intravenously injected into the gene-editing reporter mice at 2 months of age. Images show expression 3 weeks after injection. (FIG. 2A) Representative image (sagittal section) of tdTomato expression in the brains of mice given ssAAV-PHP.eb:sgRNA. Gene-editing reporter expression in the cortex (FIG. 2B) or striatum (FIG. 2C) or hippocampus (FIG. 2D) or ventral striatum (FIG. 2E) or cerebellum (FIG. 2F) in 40-μm confocal images. (FIGS. 2G-I) Gene editing reporter expression in the liver (FIG. 2G), spleen (FIG. 2H), skeletal muscle (FIG. 2I).

Continuing, FIGS. 2J-2Z, show further realization of highly efficient gene editing in adult mammalian brain in a second experiment via intravascular systemic administration of a brain-penetrable Adeno-Associated Virus (AAV). ssAAV-PHP.eb: sgRNA at 5, 1, and 0.5×10¹¹ vg/mouse was intravenously injected into the gene-editing reporter mice as described above at 2 months of age. Images show expression 3 weeks after injection. FIGS. 2J-2L are representative images (sagittal section) of tdTomato expression in the brains of mice given ssAAV-PHP.eb: sgRNA at 5, 1, and 0.5×10¹¹ vg/mouse. Gene-editing reporter expression in the cortex (FIGS. 2M and 2P), striatum (FIGS. 2N and 2Q), and hippocampus (FIGS. 2O and 2R). FIGS. 2S to 2Z show gene-editing reporter expression in the liver, spleen, skeletal muscle, heart, lung, and stomach after 1×1011 vg/mouse AAV injection.

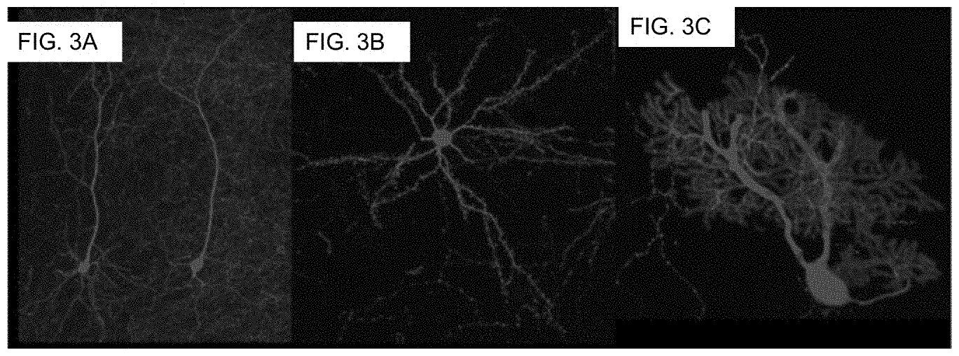

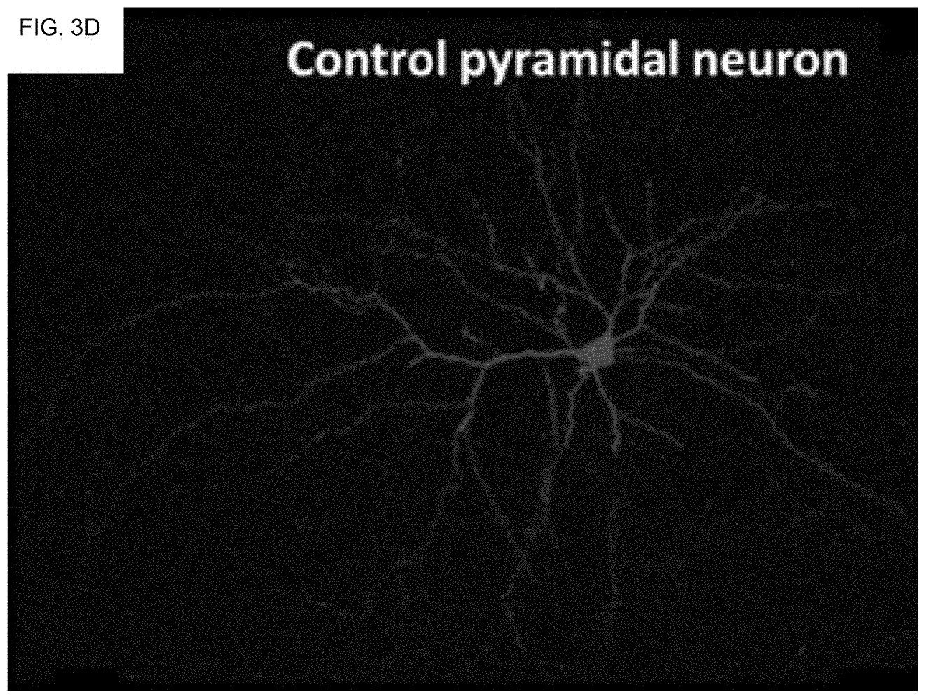

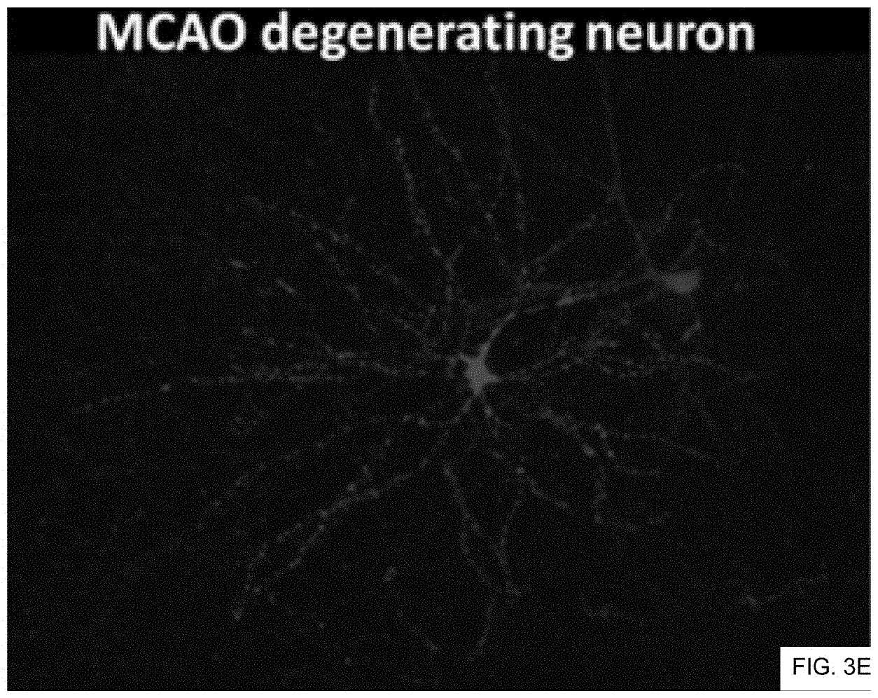

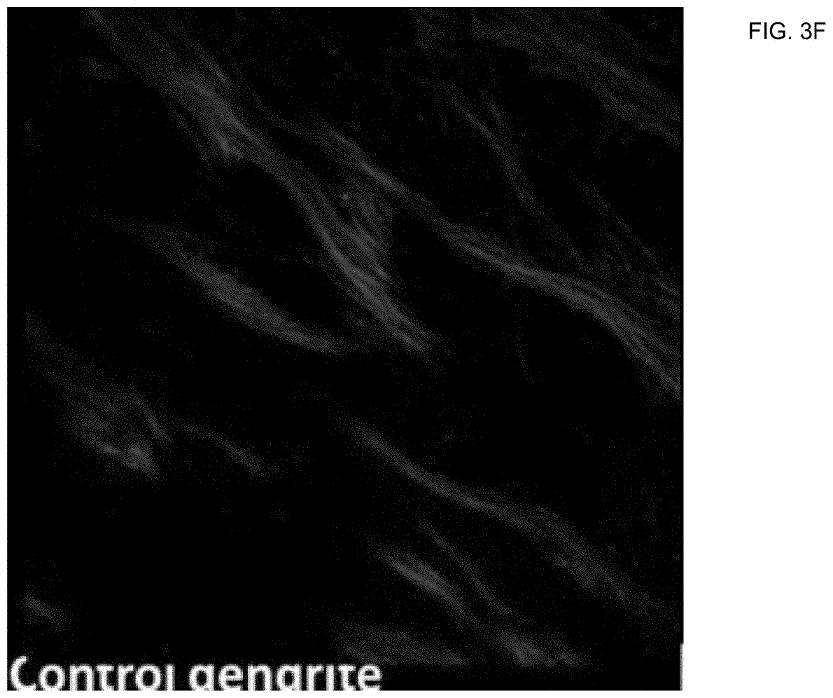

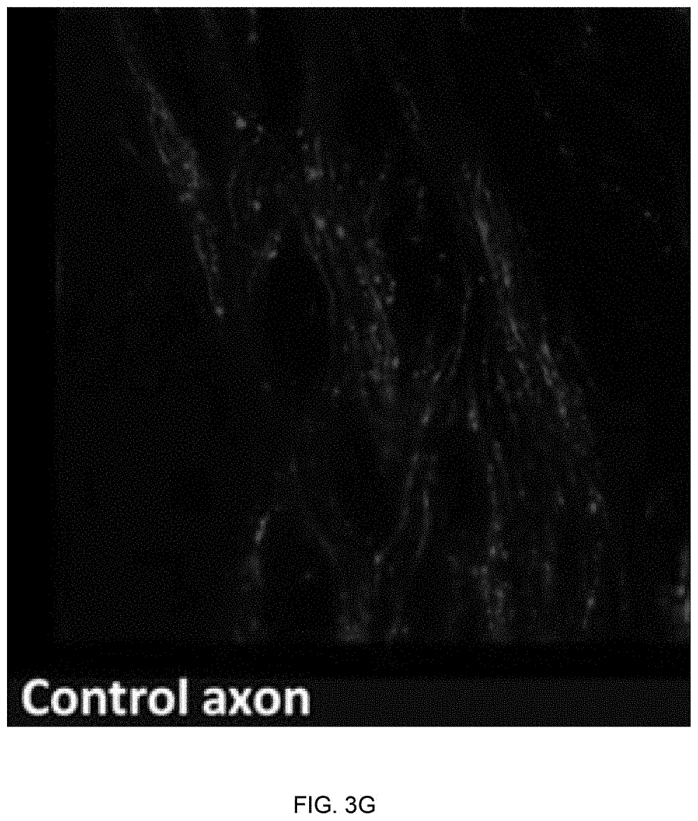

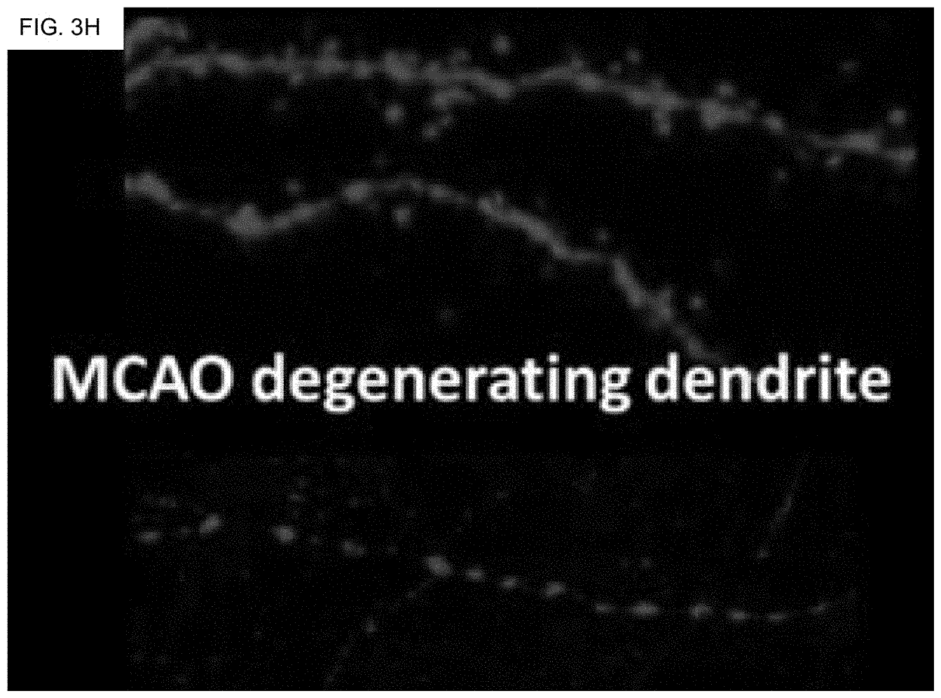

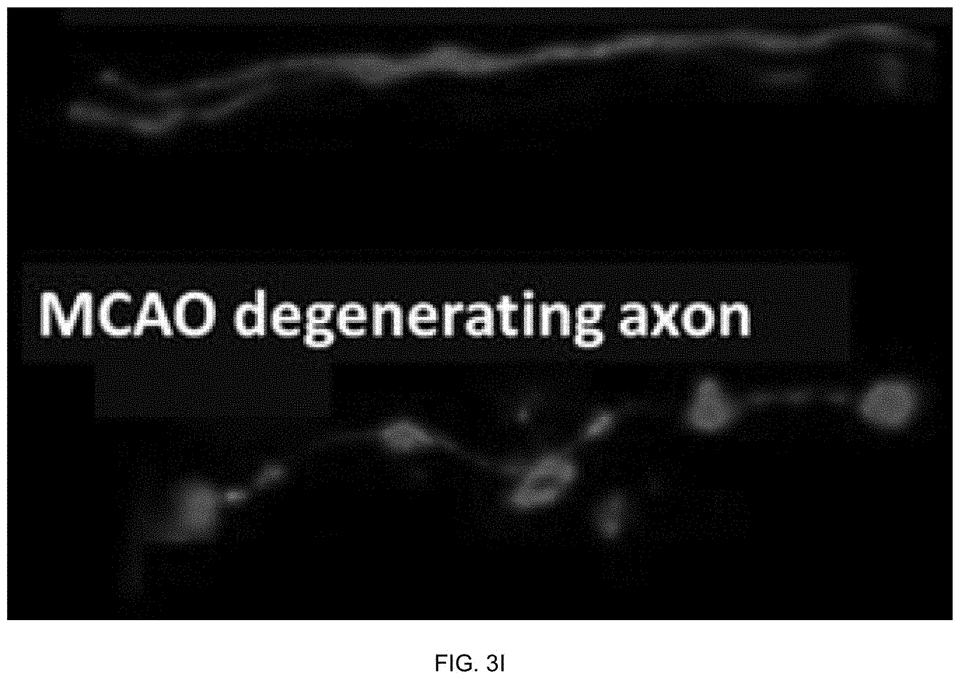

As can be seen in FIGS. 3A-3I, a second use of the technology is to visualize single-neuron morphology and neurodegeneration for the preclinical evaluation of neuroprotective therapy. FIG. 3A shows A single-labeled cortical pyramidal neuron. FIG. 3B shows a striatal medium spiny neuron, and FIG. 3C shows a cerebellum pukinje neuron shown in projections of confocal images. Contralateral control is shown in FIG. 3D and a degenerating pyramidal neuron is shown in FIG. 3E after unilateral Middle Cerebral Artery (MCA) occlusion. FIG. 3F shows a contralateral control and the degenerating axons are shown in FIG. 3G after unilateral MCA occlusion. FIG. 3H shows dendrites of a control and a degenerating striatal medium spiny neuron in MCA occlusion. FIG. 3I shows high power images of the axons of a control and a degenerating axon in MCA occlusion. The inventors noted the beaded structure, consistent with Wallerian degeneration.

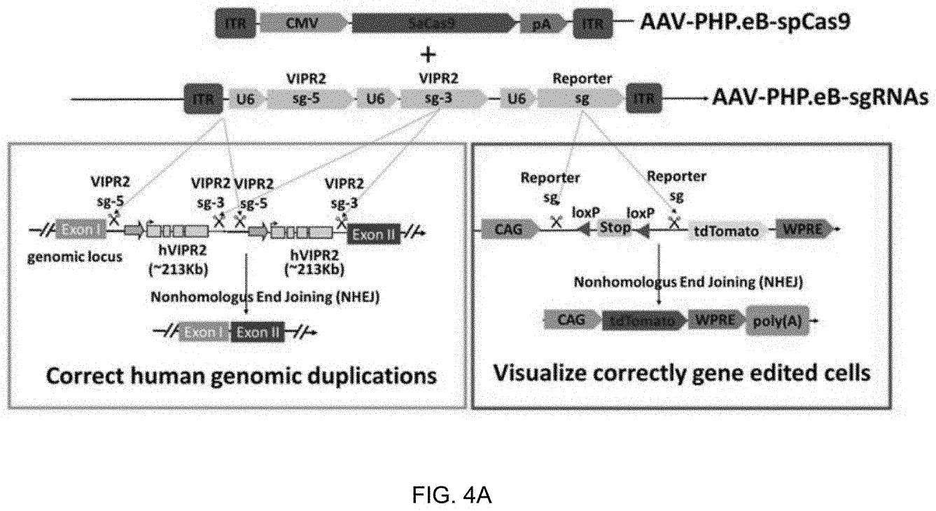

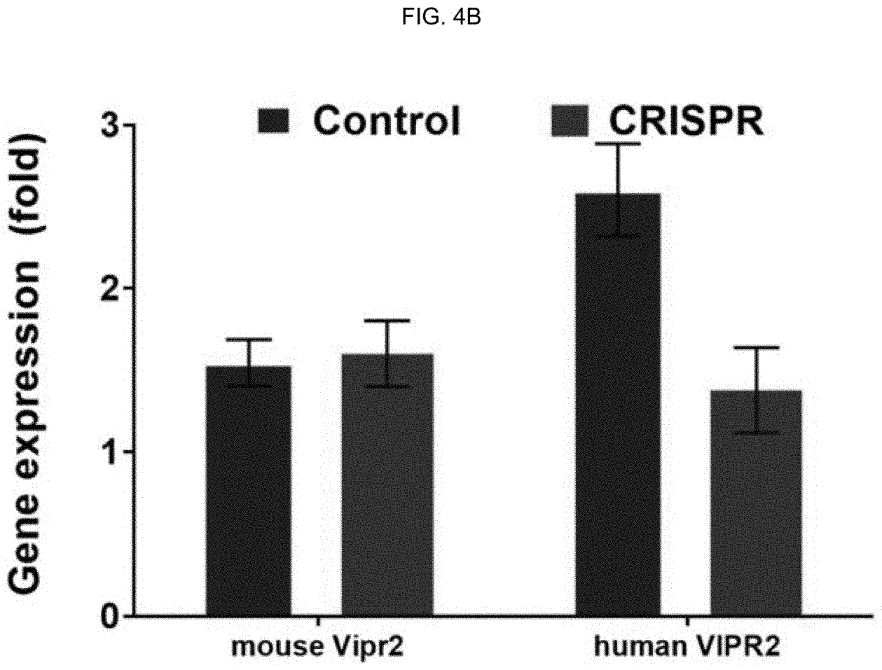

FIG. 4A shows schematics illustrating the in vivo correction of a human genomic duplication associated with the neurodevelopmental disorders via intravenous delivery of gRNA and Cas9. Using a Bacterial Artificial Chromosome (BAC) transgenic mouse model of human 7q36.3 duplication (triplication) generated in the inventors' lab, as a proof of principle, the inventors deleted the whole micro-duplicated human 7q36.3 genomic regions in the mouse model via intravascular delivery of Cas9 and sgRNAs. As shown in the FIG. 4A, exons I and II of specific genes are separated by duplicated VIPR2 gene. Paired guide RNAs (VIPR2 sg-5 and sg-3) will be designed that recognize protospacer adjacent motif on either side of human VIPR2 genomic regions. Following Cas9-mediated double-strand breaks and non-homologues end joining, the aberrant duplicated VIPR2 genomic DNAs is removed. In the same cell where VIPR2 is deleted, paired gRNAs (reporter sg-5 and sg-3) guide spCas9 to delete the STOP cassette in the genomic regions of the reporter mice. Red fluorescence protein (tdTomato) is expressed to genetically label the cells with correct VIPR2 deletion. RT-PCR in the striatum of the mice have confirmed the deletion as the level of the human VIPR2 transcripts have been reduce to 50% (FIG. 4B).

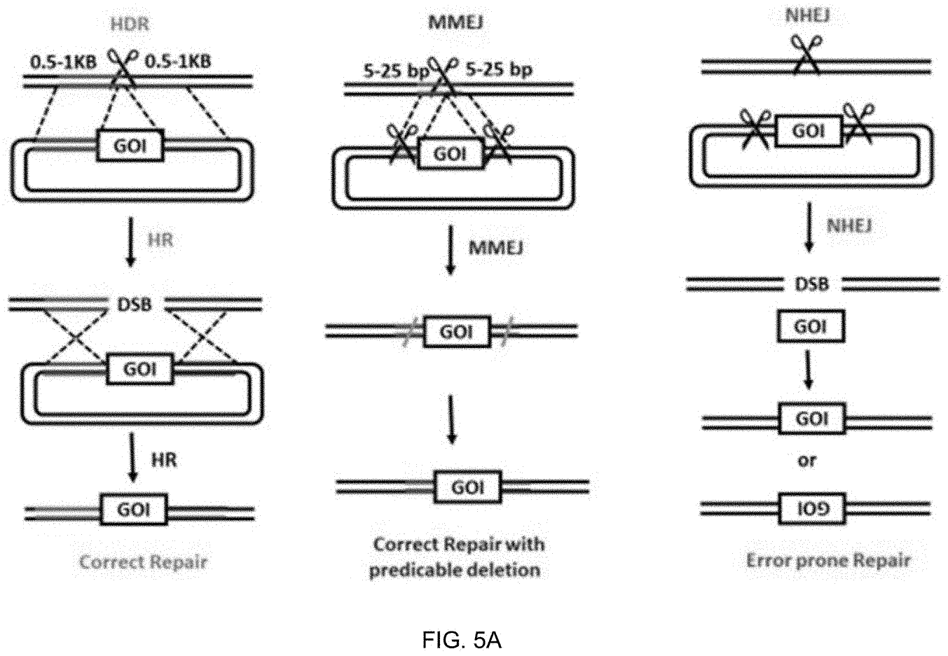

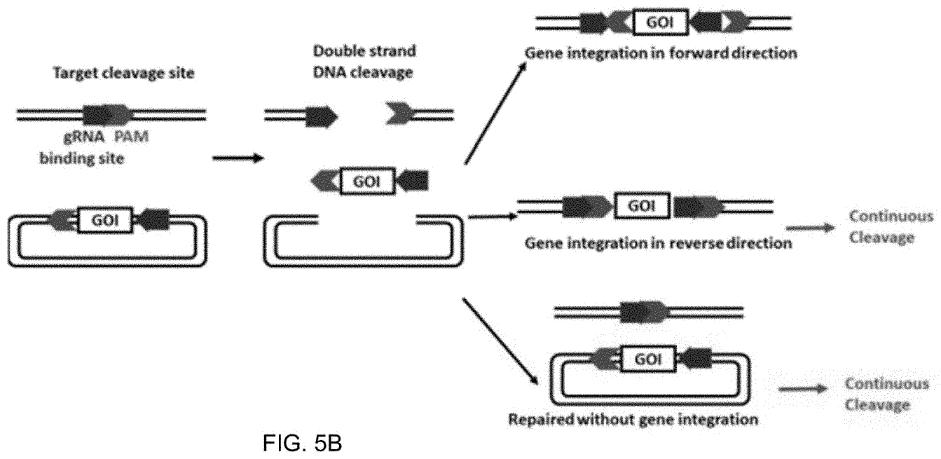

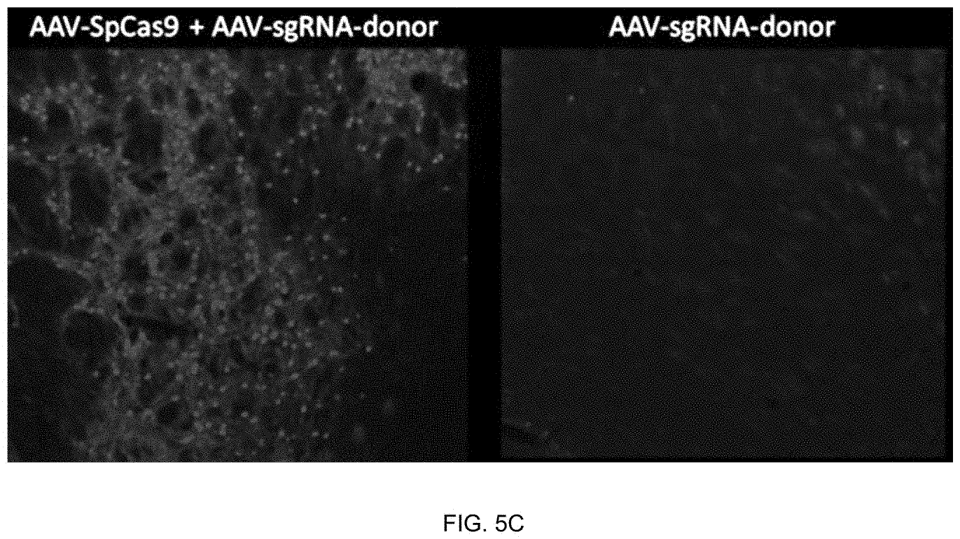

As can be seen in FIGS. 5A-5C, another use of the technology is for efficient and precise gene replacement (correction) via non-homologues end joining (non-homologues end joining) in post-mitotic neurons. DNA double-strand breaks (DSBs) are repaired by non-homologous end joining (NHEJ), and homologous recombination (HR) and a less known microhomology mediated end joining (MMEJ) (FIG. 5A). HDR HR is a precise repair pathway, which is active during the late S/G2 phases, and it requires a repair template that harbors long homology arms, however, the efficiency is very low in tissue, and especially in the post mitotic neurons. However, non-homologues end joining repair is working in any phases of cell cycles and is the major repair pathways in neurons. By taking advantage of non-homologues end joining, the inventors have designed a AAV vector to achieve precise gene replacement (insertion) (FIG. 4A, right). It is well known that non-homologues end joining connects the ends of the broken strands, can lead to unpredictable insertion and/or deletion mutations, and sometimes inserts the target sequence in the opposite direction. which is an error prone repair pathway. In order to overcome this shortcoming, the inventors have designed a “safety” mechanism. As shown in FIG. 5B, the insertion genomic fragment was flanked by incompatible sequences splitting a protospacer adjacent motif sequence and gRNA recognition site. When the non-homologues end joining mediated gene integration is in the desired direction, the protospacer adjacent motif and gRNA recognition site is destroyed. However, when gene integration is in a reverse direction, or repaired without gene integration, there will be continuous cas9 mediated cleavage until correct fragment is locked in the position. As shown in the FIG. 5C, the inventors have demonstrated that such strategy is working by inserting an IRES-Cre into the genome via intracerebral injection of AAV-sgRNA with donor sequence together with another vector to deliver Cas9.

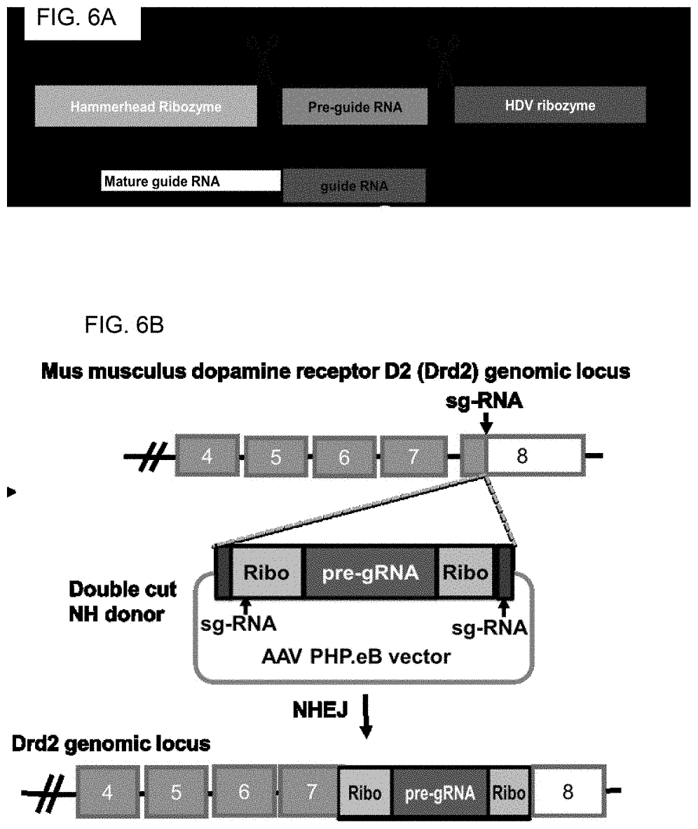

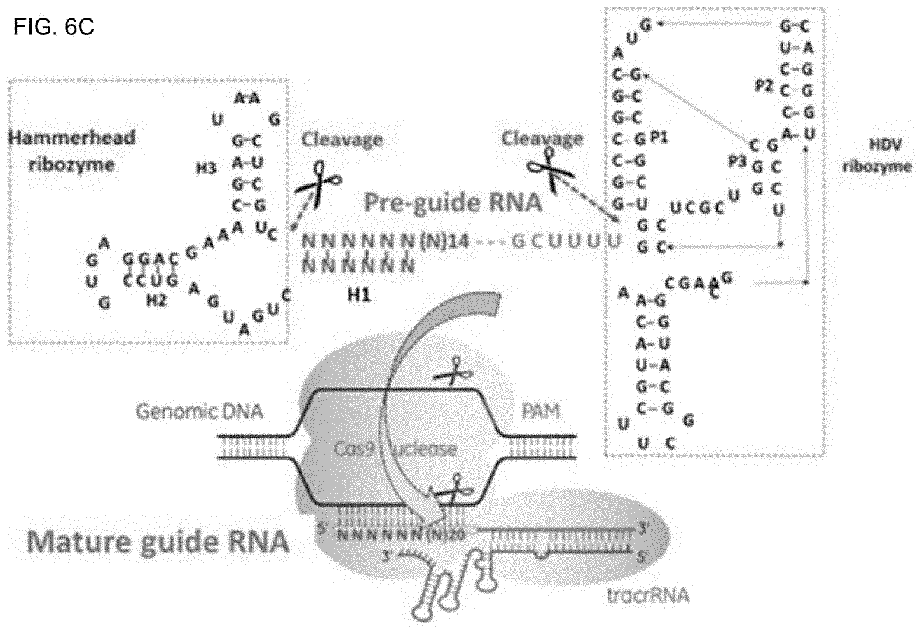

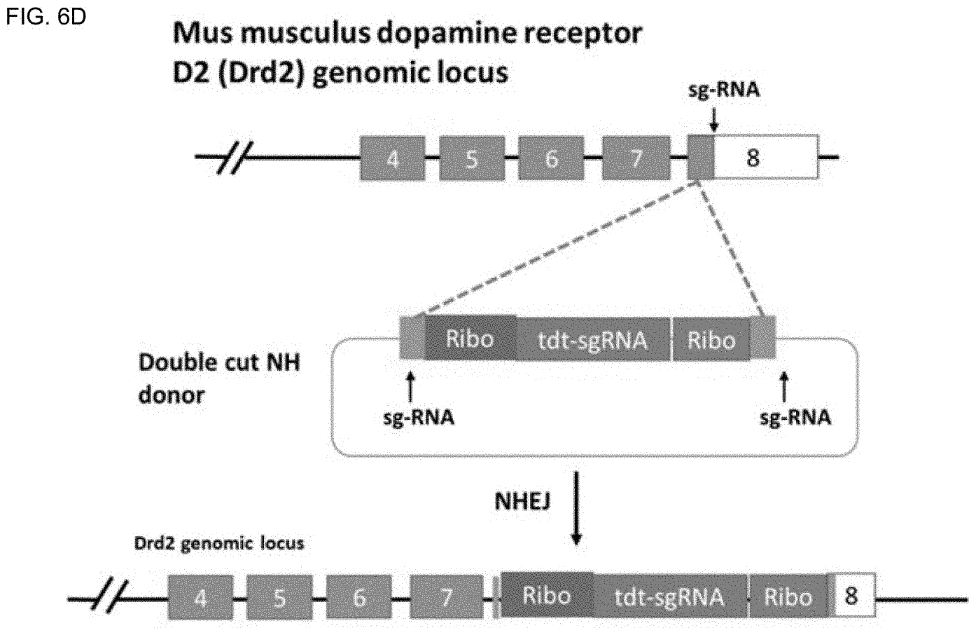

As can be seen in FIGS. 6A-6D, a further use of the technology is to achieve neuronal cell type specific gene editing. The majority of reported gene editing cases relied on gRNA production from transcriptions driven by RNA Polymerase III (Pol III) promoters, which drive RNA expression ubiquitously and constitutively. In order to directly transcribe guide RNAs from a mammalian pol II promoter, the inventors flanked the designed gRNA with two ribozymes, Hammerhead (HH) and hepatitis delta virus (HDV) ribozymes (FIGS. 6A and 6C). Ribozyme is an RNA molecule capable of acting as an enzyme to cleave RNAs. The primary transcripts will undergo self-cleavage by two flanking ribozymes to release the mature and desired gRNA for Cas9 dependent gene editing. As a proof of principle, the inventors have used the non-homologues end joining strategy as described in FIG. 4 to insert a Ribozyme-gRNA-Ribozyme sequence into 3′-UTR of the D2 receptor genomic locus in the gene editing reporter mice (FIG. 6B and 6D). Therefore, mouse endogenous D2 prompter (pol II promoter) will drive the expression of gRNA released by the self-cleavage of ribozymes, tdTomato expression will be turned on.

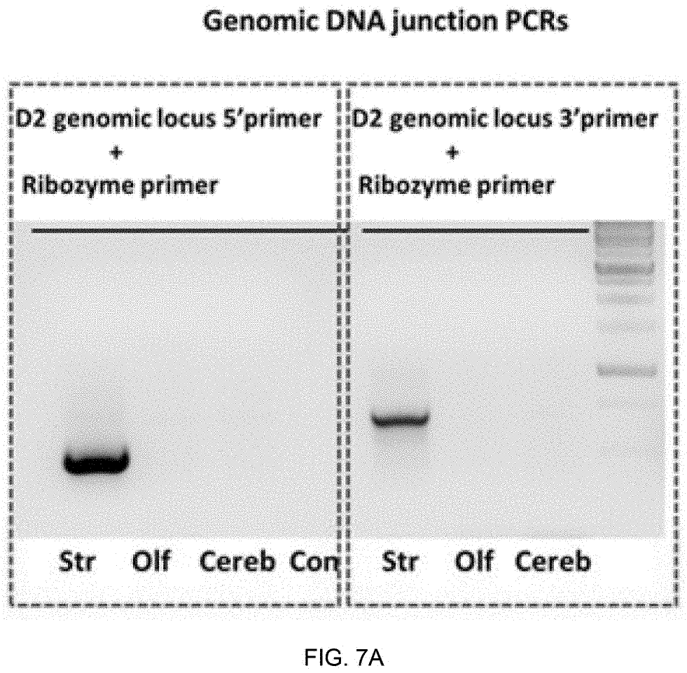

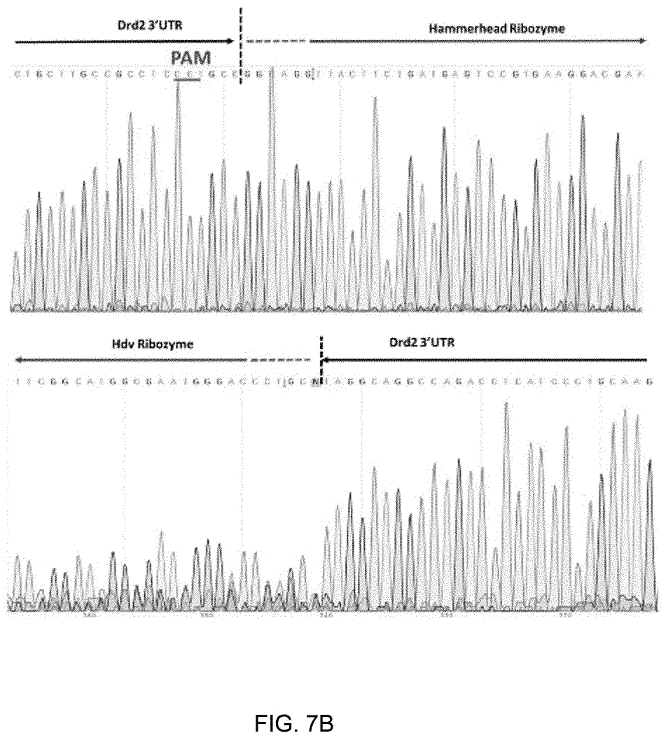

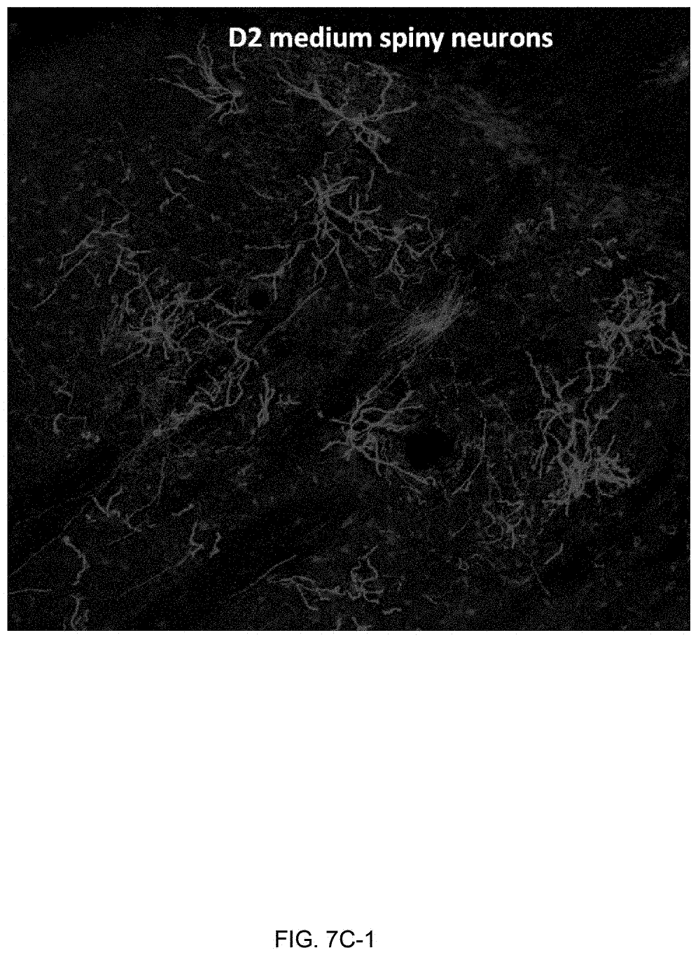

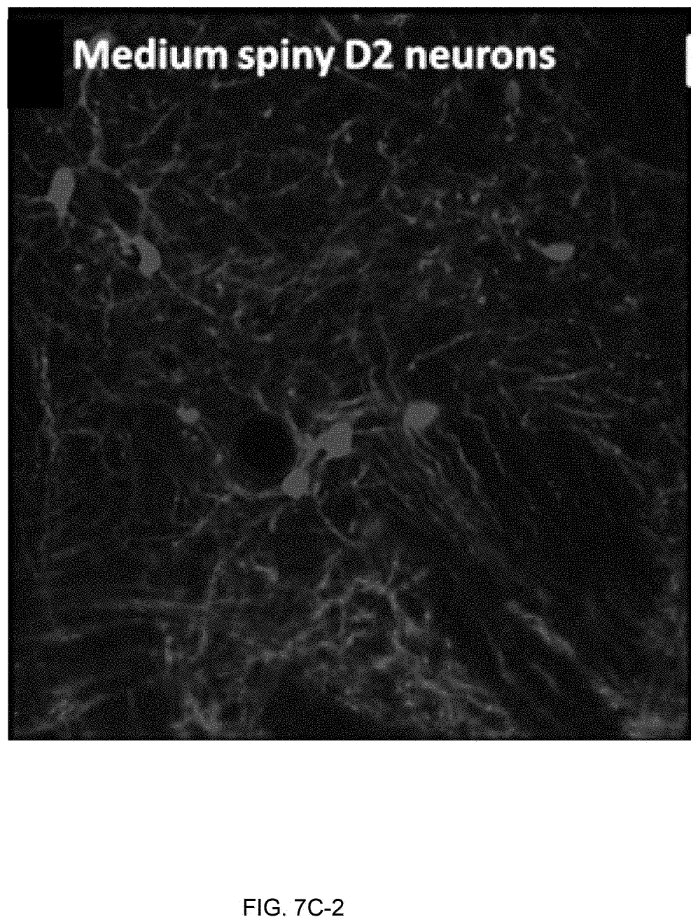

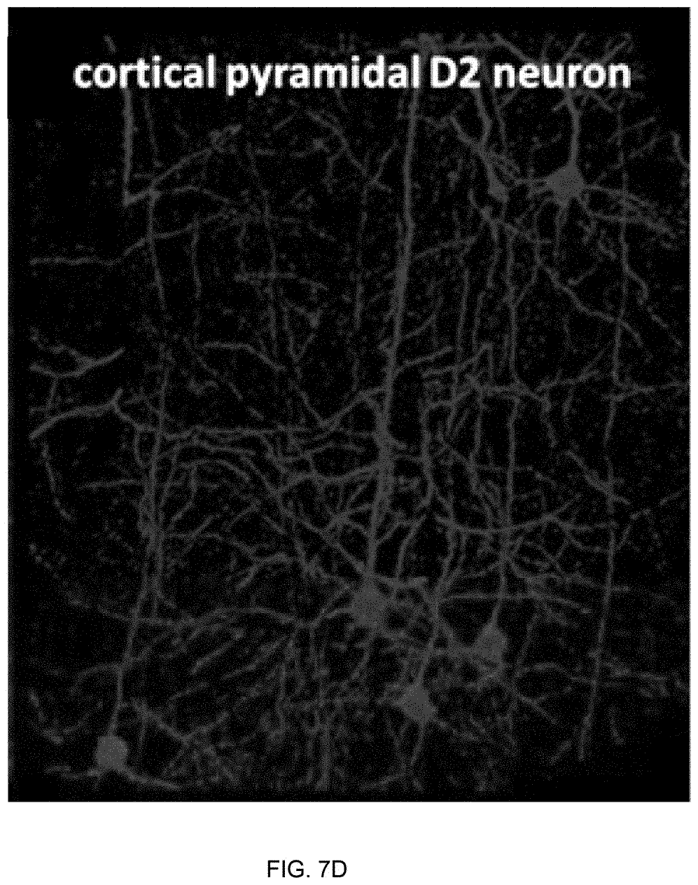

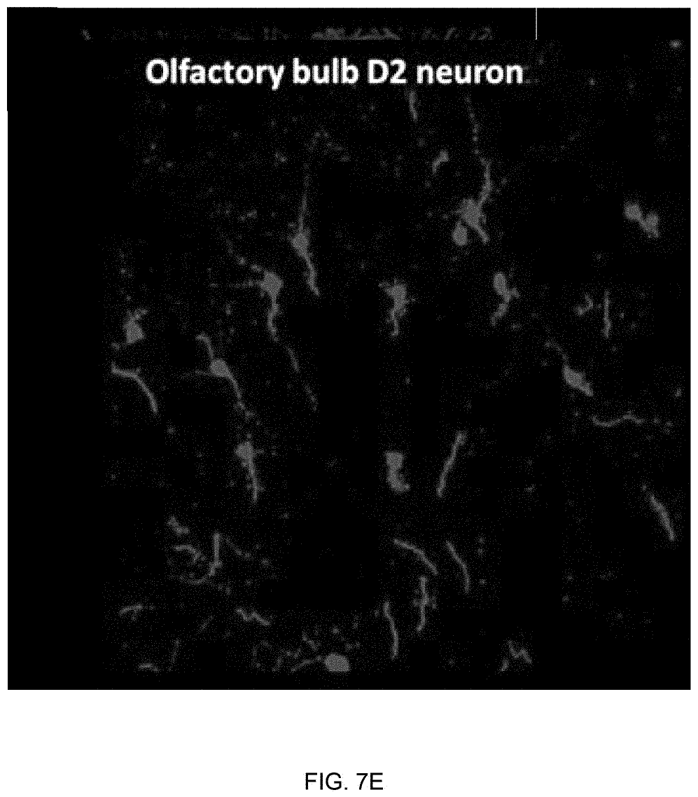

Turning next to FIGS. 7A-7E, the inventors have validated the precise gene editing to insert the Ribozyme-gRNA-Ribozyme sequence into the 3′-UTR of the D2 receptor genomic locus via junction PCRs (FIG. 7A) and genomic sequencing (FIG. 7B), and finally to use gene editing reporter mice to visualize Drd2 expressing neurons in striatum (FIG. 7C-1 and 7C-2), cortical pyrimadl neurons (FIG. 7D), and olfactory bulb neurons (FIG. 7E).

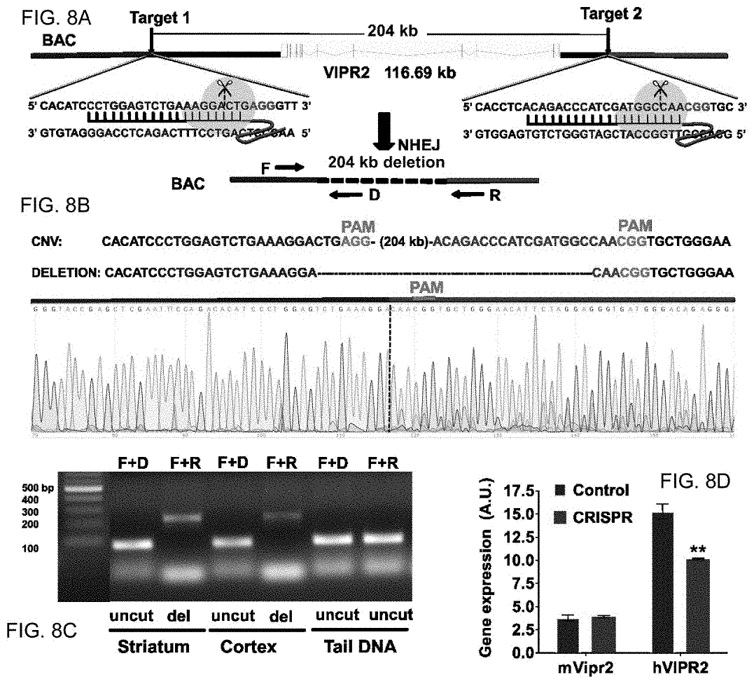

As can be shown in FIGS. 8A-8D schematic illustrating the in vivo correction of a human genomic duplication associated with the neurodevelopmental disorders via intravenous delivery of gRNA and Cas9. Using a Bacterial Artificial Chromosome (BAC) transgenic mouse model of human 7q36.3 duplication (triplication) generated in the inventors' lab, as a proof of principle, the inventors deleted the whole microduplication human 7q36.3 genomic regions in the mouse model via intravascular delivery of Cas9 and sgRNAs. FIG. 8A is a schematic overview of the strategy to delete duplicated VIPR2 CNV in vivo. The target sites on the VIPR2 BACs are indicated by arrowheads. PAM sequences are in red following the target sequence highlighted in blue. After deletion of the DNA fragment, the resulting genomic sequence is composed of the 5′ part of VIPR2 BAC (blue) and the 3′ portion of the BAC (red). The locations of human genomic fragment specific PCR primers (F, Forward; R, reverse, and D for deleted fragment) are indicated by arrows. (FIG. 8B) Sub-clone sequencing results of the PCR products amplified from the genomic DNA in striatum of the VIPR2 CNV mice (Line F) 4 weeks after the systemic delivery of the neurotropic ssAAV-PHP.eb: sgRNA at 5×1011 vg/mouse. (FIG. 8C) Agarose gel electrophoresis of the PCR analysis of genomic DNA (equal amount of the templates) from the striatum and cortex. (FIG. 8D) Striatum tissue was harvested and analyzed for reduction of VIPR2 transcripts with RT-PCR (n=4, t-test; ** p<0.01).

KITS: Any of the reagents or compositions of the invention described herein can be used together with a set of instructions, i.e., to form a kit. The kit may include instructions for use of the system as a therapy as described herein.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/We claim:

1. A method for gene editing in a vertebrate brain comprising:
intravascular administration of a brain penetrable viral vector including a target sequence in a genomic locus of interest and a CRISPR enzyme;
genomic integration via Non-Homologues End Joining (NHEJ) in post-mitotic neurons; and
editing a monopartite cell-type specific gene via NHEJ knock-in a sgRNA flanked by self-cleaving ribozymes into 3′UTR to use an endogenous promoter for sgRNA expression.

2. The method of claim 1, wherein the brain is an adult mammalian brain.

3. The method of claim 1, wherein the viral vector is an Adeno-Associated Virus (AAV).

4. The method of claim 3, wherein the AAV is one of AAV-PHP.eB, AAV1, AAV2, AAV3, AAV4, AAV5, AAV6, AAV7, AAV8, AAV9, AAV10, AAV11, AAV12, AAV2/9, AAV2/8, and AAV-DJ.

5. The method of claim 1, wherein the CRISPR enzyme is a Cas9.

6. The method of claim 5, wherein the Cas9 is one of SpCas9 and SaCas9.

7. The method of claim 1 further comprising the steps of treating a brain defect or disorder in the vertebrate.

8. The method of claim 7, wherein the brain has a genetic defect.

9. The method of claim 8, wherein the genetic defect is one of mutation, copy number variation, nucleotide repeat, duplication, triplication, and delete.

10. The method of claim 7, wherein the vertebrate has a nervous system disorder.

11. A method for gene editing in a human brain comprising:
intravascular administration of a brain penetrable viral vector including a target sequence in a genomic locus of interest and a CRISPR enzyme;
genomic integration via Non-Homologues End Joining (NHEJ) in post-mitotic neurons;
editing a monopartite cell-type specific gene via NHEJ knock-in a sgRNA flanked by self-cleaving ribozymes into 3′UTR to use an endogenous promoter for sgRNA expression; and
treating a brain defect or disorder in the vertebrate;
wherein the viral vector is an Adeno-Associated Virus (AAV) AAV-PHP.eB, the CRISPR enzyme is one of SpCas9 and SaCas9, and the brain is one of mutation, copy number variation, nucleotide repeat, duplication, triplication, and delete genetic defect.
